(19) United States
(12) Patent Application Publication
(10) Pub. No.: US 2018/0322960 A1
(43) Pub. Date: Nov. 8, 2018

(54) SYSTEM AND METHOD FOR PROCESSING GENOTYPE INFORMATION RELATING TO NSAID RISK

(71) Applicant: PROOVE BIOSCIENCES, INC., Irvine, CA (US)

(72) Inventor: Brian MESHKIN, Ladera Ranch, CA (US)

(21) Appl. No.: 15/570,315

(22) PCT Filed: Apr. 28, 2016
(86) PCT No.: PCT/US2016/029782

§ 371 Date: Oct. 27, 2017

(51) Int. Cl. G16H 70/40; G06F 19/22; G06F 19/28; G06F 19/18

(52) U.S. Cl. CPC G16H 70/40; G06F 19/18; G06F 19/28; G06F 19/22

(57) ABSTRACT

There are systems and methods for preparing or using prognostic information about NSAID mediated side effect risks. The information may include determining patient information, including DNA information, associated with a human subject; determining from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location; and determining a NSAID mediated side effect risk associated with the human subject based, at least in part, on the presence or absence of the one or more SNP diploid polymorphisms in the subject genotype.

### PRIORITY

This application claims priority to U.S. Provisional Application No. 62/153,762 entitled “System and Method for Processing Genotype Information Relating to NSAID Risk” by Brian Meshkin filed on Apr. 28, 2015, which is incorporated herein by reference in its entirety.

### CROSS-REFERENCE TO RELATED APPLICATIONS

This application is related to pending PCT Application No. TBD based on Attorney Docket No. P7916PC01 entitled “System and Method for Processing Genotype Information Relating to Drug Metabolism” by Brian Meshkin filed on Apr. 28, 2016, which is incorporated herein by reference in its entirety.

### BACKGROUND

In nature, organisms of the same species usually differ from each other in various aspects such as in their appearance or in one or more aspects of their biology. The differences are often based on genetic distinctions, some of which are called polymorphisms. Polymorphisms are often observed at the level of the whole individual (i.e., phenotype polymorphism), in variant forms of proteins and blood group substances (i.e., biochemical polymorphism), morphological features of chromosomes (i.e., chromosomal polymorphism) at the level of DNA in differences of nucleotides and/or nucleotide sequences (i.e., genetic polymorphism).

Examples of genetic polymorphisms include alleles and haplotypes. An allele is an alternative form of a gene, such as one member of a pair, that is located at a specific position on a chromosome and are known as single nucleotide polymorphisms (SNPs). A haplotype is a combination of alleles, or a combination of SNPs on the same chromosome. An example of a genetic polymorphism is an occurrence of one or more genetically alternative phenotypes in a subject due to the presence or absence of an allele or haplotype.

Genetic polymorphisms can play a role in determining differences in an individual's response to a species of drug, a drug dosage or a therapy including one drug or a combination of drugs. Pharmacogenetics and pharmacogenomics are multidisciplinary research efforts to study the relationships among genotypes, gene expression profiles, and phenotypes, as often expressed through the variability between individuals in response to the drugs taken. Since the initial sequencing of the human genome, more than a million SNPs have been identified. Some of these SNPs have been used to predict clinical predispositions or responses based upon data gathered from pharmacogenomic studies.

Chronic pain affects up to 100 million Americans (more than heart disease, cancer, and diabetes combined) and has clinical and public health implications. Nonsteroidal anti-inflammatory drugs (NSAIDs), such as aspirin, ibuprofen, naproxen, and the like, while being effective for treating and relieving pain and inflammation and for treating chronic pain conditions such as arthritis, often cause harmful side effects. NSAIDs work by inhibiting the cyclooxygenase-1 (COX-1) and cyclooxygenase-2 (COX-2) enzymes, which are involved in pro-inflammatory pathways that produce prostaglandins, prostacyclins, and thromboxanes. The number of adults taking NSAIDs in the U.S. has increased 40% from 2005 to 2010. It is not known whether physicians prescribe NSAIDs according to the intensity of the pain or by a hierarchial prescribing regimen. Use of NSAIDs however, may lead to harmful NSAID mediated side effect risks such as gastrointestinal bleeding, cardiovascular events, aspirin resistance, and Helicobacter pylori (H. pylori) infection. Each year, over 100,000 people are hospitalized with gastrointestinal complications caused by NSAID use, while an estimated 7,000 to 10,000 patients die from gastrointestingal bleeding associated with NSAID regimens. The cardiovascular safety of nonselective NSAIDs is also of concern.

While NSAIDs represent one of the most frequently prescribed drugs, inadequate prescribing practices remain frequent. Although the reported incidence of NSAID-related lower gastrointestinal complications varies, the true incidence is uncertain because patients and doctors often do not realize that there is a problem. In a postmortem examination of 713 patients, 35% of whom had used NSAIDs in the 6 months before death, 8.4% of NSAID users had nonspecific small-bowel ulcers, compared with 0.6% of nonusers, and three long-term NSAID users were found to have died from perforation of small-bowel ulcers. In the U.S., it has been estimated that about 16,500 deaths each year are related to NSAID use. Clearly, there is a need to reliably identify, prevent, and treat chronic pain conditions using NSAIDs in people with chronic pain without causing harmful side effects to the gastrointestinal tract.

It is known that first-line or maintenance NSAID medications are effective for some patients, but not others—even in instances of similar mechanisms of injury and/or etiologies of pain. However the mechanism for these differences remains somewhat unclear. Emerging scientific evidence suggests that genetic variants may play a part. Genetic factors overall are believed to account for 20% to 95% of the observed variations in drug response by individuals. In pharmacogenomics, there is a desire to identify new polymorphisms and haplotypes associated with NSAID mediated side effect risks in patients who are candidates for or taking NSAIDs. The genotype information of a patient may help a prescriber understand whether the patient is at risk for harmful NSAID mediated side effects.

A patient's genotype information is often utilized to help a prescriber decide between medications based on information associated with a patient's genetic profile (i.e., genotype or DNA information). There is a desire to utilize a patient's DNA information in determining the patient's predisposition to NSAID mediated side effect risks. There is also a desire for methods of predicting and/or diagnosing individuals exhibiting irregular predispositions to NSAID mediated side effect risks. Furthermore, there is also a desire to determine genetic information, such as polymorphisms, which may be utilized for predicting variations in NSAID mediated side effect risks among individuals. There is also a desire to implement systems processing and distributing the detected genetic information in a systematic way. Such genetic information would be useful in providing prognostic information about treatment options for a patient.

Although it is known generally that NSAID mediated side effect risks may be associated with genetics—a factor not routinely considered, there is no rigorous methodology to systematically provide doctor's with an ability to identify patients who may misuse and/or have a genetic predisposition for NSAID mediated side effect risks. Such systems and methods would be beneficial to provide information that improves accuracy in identifying patients at risk for NSAID mediated side effects.

Given the foregoing, and to address the above-described limitations, systems and methods are desired for identifying, estimating and/or determining a potential for success of an individual patient's clinical outcome in response to being prescribed a NSAID medication.

### SUMMARY

This summary is provided to introduce a selection of concepts that are further described in the Detailed Description below. The genes, polymorphisms, sequences and sequence identifiers (i.e., SEQ IDs or SEQ ID Numbers) listed or referenced herein are also described in greater detail below in the Detailed Description. This summary is not intended to identify key or essential features of the claimed subject matter. Also, this summary is not intended as an aid in determining the scope of the claimed subject matter.

The present invention meets the above-identified needs by providing systems, methods and computer readable mediums (CRMs) for preparing and utilizing prognostic information associated with a predisposition to NSAID mediated side effect risk in a patient. The prognostic information is derived from genotype information about a patient's gene profile. The genotype information may be obtained by, inter alia, assaying a sample of genetic material associated with a patient.

The systems, methods and CRMs, according to the principles of the invention, can be utilized to determine prognostic information associated with NSAID mediated side effect risk based on the patient's NSAID risk predisposition. The prognostic information may be used for addressing prescription needs directed to caring for an individual patient. It may also be utilized in managing large healthcare entities, such as insurance providers, utilizing comprehensive business intelligence systems. These and other objects are accomplished by systems, methods and CRMs directed to preparing and utilizing prognostic information associated with NSAID risk predisposition in a patient, in accordance with the principles of the invention.

According to a first principal of the invention, there is a method. The method may include facilitating a processing of and/or processing (1) data and/or (2) information and/or (3) at least one signal, the (1) data and/or (2) information and/or (3) at least one signal based, at least in part, on any combination of at least part of the following: determining patient information, including DNA information, associated with a human subject; determining from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group: ABCB1-ANC, ABCB1-HET, and ABCB1-NONA in the ABCB1 gene, COX1-ANC, COX1-HET, and COX1-NONA in the COX1 gene, PTPN11-ANC, PTPN11-HET, and PTPN11-NONA in the PTPN11 gene, NOD1-ANC, NOD1-HET, and NOD1-NONA in the NOD1 gene, TLR4-ANC, TLR4-HET, and TLR4-NONA in the TLR4 gene, CRP-ANC, CRP-HET, and CRP-NONA in the CRP gene, and COMT-ANC, COMT-HET, and COMT-NONA in the COMT gene; and determining a nonsteroidal anti-inflammatory drug (NSAID) mediated side effect risk associated with the human subject based, at least in part, on the presence or absence of the one or more SNP diploid polymorphisms in the subject genotype.

The method may also include wherein the (1) data and/or (2) information and/or (3) at least one signal are further based, at least in part, on any combination the following: determining from the DNA information whether a subject genotype of the human subject includes at least two CYP haplotype polymorphisms by detecting, utilizing a detection technology and the DNA information, a presence or absence of the at least two CYP haplotype polymorphisms in the subject genotype, wherein at least one or more CYP haplotype polymorphisms are selected from the CYP2C8 haplotype group including normal function CYP2C8 star alleles and reduced function CYP2C8 star alleles, wherein at least one or more CYP haplotype polymorphisms are selected from the CYP2C9 haplotype group including normal function CYP2C9 star alleles, reduced function CYP2C9 star alleles and null function CYP29 star alleles, determining a comparing of a region, including the one or more SNP diploid polymorphisms, of the subject genotype with a corresponding region of a predetermined reference genotype, wherein characteristics of the corresponding region of the reference genotype are based upon a predetermined population norm; determining prognostic information associated with the human subject based on the determined NSAID mediated side effect risk; and determining a therapy for the human subject based on the determined prognostic information associated with the human subject, wherein the method for determining the NSAID risk associated with the human subject, is an ex vivo method. The one or more SNP diploid polymorphisms may include at least any number of two through seven SNP diploid polymorphisms from the SNP diploid group.

According to a second principal of the invention, there is an apparatus. The apparatus may include any combination of at least one processor; and at least one memory including computer program code for one or more programs, the at least one memory and the computer program code configured to, with the at least one processor, cause the apparatus to perform at least the following, determine patient information, including DNA information, associated with a human subject; determine from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group: ABCB1-ANC, ABCB1-HET, and ABCB1-NONA in the ABCB1 gene, COX1-ANC, COX1-HET, and COX1-NONA in the COX1 gene, PTPN11-ANC, PTPN11-HET, and PTPN11-NONA in the PTPN11 gene, NOD1-ANC, NOD1-HET, and NOD1-NONA in the NOD1 gene, TLR4-ANC, TLR4-HET, and TLR4-NONA in the TLR4 gene, CRP-ANC, CRP-HET, and CRP-NONA in the CRP gene, and COMT-ANC, COMT-HET, and COMT-NONA in the COMT gene; and determine a nonsteroidal anti-inflammatory drug (NSAID) mediated side effect risk associated with the human subject based, at least in part, on the presence or absence of the one or more SNP diploid polymorphisms in the subject genotype.

According to a third principal of the invention, there is a non-transitory computer readable medium. The medium may store any combination of computer readable instructions that when executed by at least one processor perform a method, the method comprising facilitating a processing of and/or processing (1) data and/or (2) information and/or (3) at least one signal, the (1) data and/or (2) information and/or (3) at least one signal based, at least in part, on any combination of the following: determining patient information, including DNA information, associated with a human subject; determining from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group: ABCB1-ANC, ABCB1-HET, and ABCB1-NONA in the ABCB1 gene, COX1-ANC, COX1-HET, and COX1-NONA in the COX1 gene, PTPN11-ANC, PTPN11-HET, and PTPN11-NONA in the PTPN11 gene, NOD1-ANC, NOD1-HET, and NOD1-NONA in the NOD1 gene, TLR4-ANC, TLR4-HET, and TLR4-NONA in the TLR4 gene, CRP-ANC, CRP-HET, and CRP-NONA in the CRP gene, and COMT-ANC, COMT-HET, and COMT-NONA in the COMT gene; and determining a nonsteroidal anti-inflammatory drug (NSAID) mediated side effect risk associated with the human subject based, at least in part, on the presence or absence of the one or more SNP diploid polymorphisms in the subject genotype.

The above summary is not intended to describe each embodiment or every implementation of the present invention. Further features, their nature and various advantages are made more apparent from the accompanying drawings and the following examples and embodiments.

### BRIEF DESCRIPTION OF THE DRAWINGS

Features and advantages of the present invention become more apparent from the detailed description, set forth below, when taken in conjunction with the drawings. In the drawings, like reference numbers indicate identical or functionally similar elements. Additionally, a left-most digit of a reference number identifies a drawing in which the reference number first appears. In addition, it should be understood that the drawings in the figures which highlight an aspect, methodology, functionality and/or advantage of the present invention, are presented for example purposes only. The present invention is sufficiently flexible such that it may be implemented in ways other than shown in the accompanying figures.

FIG. 1 is a block diagram illustrating an assay system which may be utilized for preparing genotype information from a sample of genetic material, according to an example;

FIG. 2 is a block diagram illustrating a prognostic information system which may be utilized for preparing and/or utilizing prognostic information utilizing the genotype information prepared using the assay system of FIG. 1, according to an example;

FIG. 3 is a flow diagram illustrating a prognostic information process for identifying a risk to a patient utilizing the assay system of FIG. 1 and the prognostic information system of FIG. 2, according to an example; and

FIG. 4 is a block diagram illustrating a computer system providing a platform for the assay system of FIG. 1 or the prognostic information system of FIG. 2, according to various examples.

### DETAILED DESCRIPTION

The present invention is useful for preparing and/or utilizing prognostic information about a patient. The prognostic information may be utilized to determine an appropriate therapy for the patient based on their genotype and phenotype information and identify their genetic predisposition to risk of NSAID mediated side effects. The genetic predisposition may be associated with the selection of a NSAID medication, a dosage of the NSAID medication and the utilization of the NSAID medication in a regimen for treating the patient's medical condition.

The prognostic information may also be utilized for determining dose adjustments that may help a prescriber understand why a patient is or is not responding to a NSAID medication dosage, such as an “average” dose. The prognostic information may also be utilized by a prescriber to decide between medications based on the patient's genetic predisposition to NSAID mediated side effect risk. The prognostic information may also be utilized for predicting and/or diagnosing individuals exhibiting a regular or irregular predisposition to NSAID mediated side effect risk. Such genetic information can be very useful in providing prognostic information about treatment options for a patient. The patient may be associated with a medical condition. The patient may also have already been prescribed a medication for treating the medical condition. The present invention has been found to be advantageous for determining a treatment for a patient who may have a regular or irregular predisposition to NSAID mediated side effect risk. While the present invention is not necessarily limited to such applications, various aspects of the invention may be appreciated through a discussion of the various examples in this context, as illustrated through the examples below.

For simplicity and illustrative purposes, the present invention is described by referring mainly to embodiments, principles and examples thereof. In the following description, numerous specific details are set forth in order to provide a thorough understanding of the examples. It is readily apparent however, that the embodiments may be practiced without limitation to these specific details. In other instances, some embodiments have not been described in detail so as not to unnecessarily obscure the description. Furthermore, different embodiments are described below. The embodiments may be used or performed together in different combinations.

The operation and effects of certain embodiments can be more fully appreciated from the examples described below. The embodiments on which these examples are based are representative only. The selection of embodiments is to illustrate the principles of the invention and does not indicate that variables, functions, conditions, techniques, configurations and designs, etc., which are not described in the examples are not suitable for use, or that subject matter not described in the examples is excluded from the scope of the appended claims and their equivalents. The significance of the examples can be better understood by comparing the results obtained therefrom with potential results which can be obtained from tests or trials that may be or may have been designed to serve as controlled experiments and provide a basis for comparison.

Before the systems and methods are described, it is understood that the invention is not limited to the particular methodologies, protocols, systems, platforms, assays, and the like which are described, as these may vary. It is also to be understood that the terminology used herein is intended to describe particular embodiments of the present invention, and is in no way intended to limit the scope of the present invention as set forth in the appended claims and their equivalents.

Throughout this disclosure, various publications, such as patents and published patent specifications, are referenced by an identifying citation. The disclosures of these publications are hereby incorporated by reference in their entirety into the present disclosure in order to more fully describe the state of the art to which the invention pertains.

The practice of the present invention employs, unless otherwise indicated, conventional techniques of molecular biology, microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al., U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)); MANIPULATING THE MOUSE EMBRYO: A LABORATORY MANUAL 3rd edition (Cold Spring Harbor Laboratory Press, Cold Spring Harbor, N.Y. (2002)).

### Definitions

As used herein, certain terms have the following defined meanings. As used herein, the singular form “a,” “an” and “the” includes the singular and plural references unless the context clearly dictates otherwise. For example, the term “a cell” includes a single cell and a plurality of cells, including mixtures thereof.

As used herein, the terms “based on,” “comprises,” “comprising,” “includes,” “including,” “has,” “having” or any other variation thereof, are intended to cover a non-exclusive inclusion. For example, a system, process, method, article, or apparatus that comprises a list of elements is not necessarily limited to only those elements but may include other elements not expressly listed or inherent to such system, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

All numerical designations, e.g., pH, temperature, time, concentration, and molecular weight, including ranges, are approximations which may be varied (+) or (−) by minor increments, such as, of 0.1. It is to be understood, although not always explicitly stated, that all numerical designations are preceded by the term “about”. The term “about” also includes the exact value “X” in addition to minor increments of “X” such as “X+0.1” or “X-0.1.” It also is to be understood, although not always explicitly stated, that the reagents described herein are merely exemplary and that equivalents of such are known to those of ordinary skill in the art.

The term “allele” which is used interchangeably herein with the term “allelic variant” refers to alternative forms of a gene or any portions thereof. Alleles may occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions and insertions of nucleotides. An allele of a gene can also be an ancestral form of a gene or a form of a gene containing a mutation.

The term “haplotype” refers to a combination of alleles on a chromosome or a combination of SNPs within an allele on one chromosome. The alleles or SNPs may or may not be at adjacent locations (loci) on a chromosome. A haplotype may be at one locus, at several loci or an entire chromosome.

The term “ancestral,” when applied to describe an allele in a human, refers to an allele of a gene that is the same or nearest to a corresponding allele appearing in the corresponding gene of the chimpanzee genome. Often, but not always, a human ancestral allele is the most prevalent human allelic variant appearing in nature—i.e., the allele with the highest gene frequency in a population of the human species.

The term “wild-type,” when applied to describe an allele, refers to an allele of a gene which, when it is present in two copies in a subject, results in a wild-type phenotype. There can be several different wild-type alleles of a specific gene. Also, nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region may include, for example, a single nucleotide polymorphism (SNP), the identity of which differs in the different alleles by a single nucleotide at a locus in the polymorphic region of the gene. In another example, a polymorphic region may include a deletion or substitution of one or more nucleotides at a locus in the polymorphic region of the gene.

The expression “amplification of polynucleotides” includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, a PCR procedure is a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively, the amplified sequence(s) may be cloned prior to sequence analysis. Methods for direct cloning and sequence analysis of enzymatically amplified genomic segments are known in the art.

The term “encode,” as it is applied to polynucleotides, refers to a polynucleotide which is said to “encode” a polypeptide. The polynucleotide is transcribed to produce mRNA, which is then translated into the polypeptide and/or a fragment thereof by cell machinery. An antisense strand is the complement of such a polynucleotide, and the encoding sequence can be deduced therefrom.

As used herein, the term “gene” or “recombinant gene” refers to a nucleic acid molecule comprising an open reading frame and including at least one exon and optionally an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A “related” or “homologous” sequence shares identity with a comparative sequence, such as 100%, at least 99%, at least 95%, at least 90%, at least 80%, at least 70%, at least 60%, at least 50%, at least 40%, at least 30%, at least 20%, or at least 10%. An “unrelated” or “non-homologous” sequence shares less identity with a comparative sequence, such as less than 95%, less than 90%, less than 80%, less than 70%, less than 60%, less than 50%, less than 40%, less than 30%, less than 20%, or less than 10%.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “isolated” as used herein with respect to nucleic acids, such as DNA or RNA, refers to molecules separated from other DNAs or RNAs, respectively, which are present in a natural source of a macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the term “nucleic acid” refers to polynucleotides such as deoxyribonucleic acid (DNA), and, where appropriate, ribonucleic acid (RNA). The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides.

Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine or A,” “cytidine or C,” “guanosine or G,” and “thymidine or T” are used. It is understood that if the nucleic acid is RNA, it includes nucleotide(s) having a uracil base that is “uridine or U”.

The terms “oligonucleotide” or “polynucleotide,” or “portion,” or “segment” thereof refer to a stretch of polynucleotide residues which may be long enough to use in PCR or various hybridization procedures to identify or amplify identical or related parts of mRNA or DNA molecules. The polynucleotide compositions described herein may include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications can include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). This may also include synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The phrase “genetic profile” is used interchangeably with “genotype information” and refers to part or all of an identified genotype of a subject and may include one or more polymorphisms in one or more genes of interest. A genetic profile may not be limited to specific genes and polymorphisms described herein, and can include any number of other polymorphisms, gene expression levels, polypeptide sequences, or other genetic markers that are associated with a subject or patient.

The term “patient” refers to an individual waiting for or under medical care and treatment, such as a treatment for medical condition. While the disclosed methods are designed for human patients, such methods are applicable to any suitable individual, which includes, but is not limited to, a mammal, such as a mouse, rat, rabbit, hamster, guinea pig, cat, dog, goat, cow, horse, pig, and simian. Human patients include male and female patients of any ethnicity. The term “treating” as used herein is intended to encompass curing as well as ameliorating at least one symptom of a condition or disease.

The nucleic acid codes utilized herein include: A for Adenine, C for Cytosine, G for Guanine, T for Thymine, U for Uracil, R for A or G, Y for C, T or U, K for G, T or U, and M for A or C.

As used herein, the terms “drug,” “medication,” and “therapeutic compound” or “compound” are used interchangeably and refer to any chemical entity, pharmaceutical, drug, biological, and the like that can be used to treat or prevent a disease, illness, condition, or disorder of bodily function. A drug may comprise both known and potentially therapeutic compounds. A drug may be determined to be therapeutic by screening using the screening known to those having ordinary skill in the art. A “known therapeutic compound” or “medication” refers to a therapeutic compound that has been shown (e.g., through animal trials or prior experience with administration to humans) to be effective in such treatment. Examples of drugs include, but are not limited to peptides, polypeptides, synthetic organic molecules, naturally occurring organic molecules, nucleic acid molecules, and combinations thereof.

The biological basis for an outcome in a specific patient following a treatment with an NSAID medication is subject to, inter alia, the patient's genetic predisposition to NSAID mediated side effect risk. It has been determined that select polymorphisms of a patient, including single nucleotide permutations, haplotypes and phenotypes may be utilized to generate genotype information. The genotype information may be utilized to generate prognostic information. The prognostic information may be utilized in determining treatment options for the patient. The prognostic information is based on the patient's genetic predisposition to NSAID mediated side effect risk. The prognostic information may also be utilized in determining an expected outcome of a treatment of an individual, such as a treatment with an NSAID medication.

When a genetic marker such as a polymorphism is used as a basis for determining a treatment for a patient, as described herein, the genetic marker may be measured before or during treatment. The prognostic information obtained may be used by a clinician in assessing any of the following: (a) a probable or likely suitability of an individual to initially receive NSAID medication treatment(s); (b) a probable or likely unsuitability of an individual to initially receive NSAID medication treatment(s); (c) a responsiveness to NSAID medication treatment; (d) a probable or likely suitability of an individual to continue to receive treatment(s); (e) a probable or likely unsuitability of an individual to continue to receive treatment(s); (f) adjusting dosage; (g) predicting likelihood of clinical benefits. As understood by one of skill in the art, measurement of a genetic marker or polymorphism in a clinical setting can be an indication that this parameter may be used as a basis for initiating, continuing, adjusting and/or ceasing administration of NSAID medication treatment, such as described herein.

Select polymorphisms, including SNPs and/or haplotypes, have been identified which may be utilized for providing prognostic information, according to the principles of the invention. These findings were correlated with various magnitudes of a positive or negative predispositions to NSAID mediated side effect risk. Accordingly, assaying the genotype at these markers may be utilized to generate prognostic information which may be utilized to predict the expected outcome of treating the patient with an NSAID pain medication based on the expected predisposition of the patient to NSAID mediated side effect risk. Clinicians prescribing NSAID medication and other medications may utilize the prognostic information to improve therapeutic decisions and to avoid treatment failures.

Many of the known human single nucleotide permutations (SNPs) are catalogued by the National Center for Biotechnology Information (NCBI) in the Reference SNP (i.e., “refSNP”) database maintained by NCBI. The Reference SNP database is a polymorphism database (dbSNP) which includes single nucleotide polymorphisms and related polymorphisms, such as deletions and insertions of one or more nucleotides. The database is a public-domain archive maintained by NCBI for a broad collection of simple genetic polymorphisms and can be accessed at http://www.ncbi.nlm.nih.gov/snp.

A number of patients have experienced adverse outcomes, including gastrointestinal bleeding, cardiovascular events, aspirin resistance, and H. pylori infection leading to gastric or rectal cancer with NSAID use. Numerous investigations have demonstrated that this phenomenon may be, in part, attributed to the broad variability in individual response profiles and to genetic polymorphisms in candidate genes involved in immunological and inflammatory signaling pathways. Using these polymorphisms to identify patients at risk of adverse events would play an important role in modulating NSAID risk factors. Additionally, a characterization of a patient's metabolic profile for NSAID-induced hepatotoxicity would add crucial information to a patient's clinical care as well.

DNA polymorphisms have been identified which may be utilized according to the principles of the invention include SNPs and haplotypes associated with genetic markers in several genes. The genes include the respective genes encoding the ATP binding cassette sub-family B member 1 (ABCB1), Cyclooxygenase-1 (COX-1), Tyrosine-protein phosphatase non-receptor type 11 (PTPN11) (also known as protein-tyrosine phosphatase 1D (PTP-1D) or protein-tyrosine phosphatase 2C (PTP-2C)), Nucleotide-binding oligomerization domain-containing protein 1 (NOD1), Toll-like receptor 4 (TLR4), C-reactive protein (CRP), Catechol 0-Methyltransferase (COMT), Cytochrome P450 2C8 (CYP2C8), and Cytochrome P450 2C9 (CYP2C9).

The panel of genetic markers describe above can be used to predict several risk factors with NSAIDs. This risk test focuses on SNPs in candidate genes involved with innate immunity and inflammation (e.g., COX-1, TLR4, CRP, NOD1, PTPN11, COMT, and genes involved in NSAID metabolism) and efflux (e.g., CYP2C8, CYP2C9, and ABCB1). The risk of a NSAID mediated side effect can be assessed using the polymorphisms found in these genes and, optionally, as well as by characterizing the patient's metabolic profile, as genetic polymorphisms in metabolizing enzymes can be regarded as one of the causes of inter-individual variation in response to medications and in development of adverse reactions.

For example, a method provided by the invention is a diagnostic method for determining the NSAID risk associated with a patient which method is not practised on the patient's body, i.e. is an ex vivo diagnostic method. The method may involve determining patient information which may be obtained by assaying a sample of genetic material associated with the patient. The method does not involve obtaining the sample from the patient's body. The invention also provides uses of the systems and methods, for example of the diagnostic assays, for determining the OD risk associated with a patient.

The DNA polymorphisms which have been identified as active for predicting a genetic predisposition to risk of NSAID-related gastrointestinal complications are SNP Diploid Polymorphisms. In the identified SNP diploid polymorphisms, the predisposition to risk of NSAID-related gastrointestinal complications varies depending upon the active allele of a SNP in a chromosome of a gene as well as the zygosity of the SNP diploid at the locus of the SNP on the chromosome. The SNP diploid polymorphisms identified as predisposition to risk of NSAID-related gastrointestinal complications are listed in Table 1 below.

TABLE 1*
Identification of SNP Diploid Polymorphisms @

| | | SNP | | | |
|---|---|---|---|---|---|
| | | Diploid | | DNA Context Sequence for | |
| No. | rs# | ID** | Zygosity | Active SNP(s)*** | SEQ ID |
| 1 | rs1045642 | ABCB1- | homozygous | GCCGGGTGGTGTCACAGGAAGAGAT[C] | SEQ ID |
| | | ANC | | GTGAGGGCAGCAAAGGAGGCCAACA | No: 1 |
| 2 | rs1045642 | ABCB1- | heterozygous | GCCGGGTGGTGTCACAGGAAGAGAT[C/T] | SEQ ID |
| | | HET | | GTGAGGGCAGCAAAGGAGGCCAACA | No: 2 |
| 3 | rs1045642 | ABCB1- | homozygous | GCCGGGTGGTGTCACAGGAAGAGAT[T] | SEQ ID |
| | | NONA | | GTGAGGGCAGCAAAGGAGGCCAACA | No: 3 |
| 4 | rs1330344 | COX1- | homozygous | GAAACACTTGTGTGGCCCTGGCACT[G] | SEQ ID |
| | | ANC | | ATGGGAAGAGCCTTCACCTCAGAAT | No: 4 |
| 5 | rs1330344 | COX1- | heterozygous | GAAACACTTGTGTGGCCCTGGCACT[A/G] | SEQ ID |
| | | HET | | ATGGGAAGAGCCTTCACCTCAGAAT | No: 5 |
| 6 | rs1330344 | COX1- | homozygous | GAAACACTTGTGTGGCCCTGGCACT[A] | SEQ ID |
| | | NONA | | ATGGGAAGAGCCTTCACCTCAGAAT | No: 6 |
| 7 | rs2301756 | PTPN11- | homozygous | ATGACCACTAAACTTCTTAAATGAG[C] | SEQ ID |
| | | ANC | | CCACAGTCCTTTAGAGACAAATGCC | No: 7 |
| 8 | rs2301756 | PTPN11- | heterozygous | ATGACCACTAAACTTCTTAAATGAG[C/T] | SEQ ID |
| | | HET | | CCACAGTCCTTTAGAGACAAATGCC | No: 8 |
| 9 | rs2301756 | PTPN11- | homozygous | ATGACCACTAAACTTCTTAAATGAG[T] | SEQ ID |
| | | NONA | | CCACAGTCCTTTAGAGACAAATGCC | No: 9 |
| 10 | rs7789045 | NOD1- | homozygous | TTGCTGACTGGTGGTCTCTTCCAGC[A] | SEQ ID |
| | | ANC | | GACTTGAAGCTCCCTGAGGGCAGGA | No: 10 |
| 11 | rs7789045 | NOD1- | heterozygous | TTGCTGACTGGTGGTCTCTTCCAGC[A/T] | SEQ ID |
| | | HET | | GACTTGAAGCTCCCTGAGGGCAGGA | No: 11 |
| 12 | rs7789045 | NOD1- | homozygous | TTGCTGACTGGTGGTCTCTTCCAGC[T] | SEQ ID |
| | | NONA | | GACTTGAAGCTCCCTGAGGGCAGGA | No: 12 |
| 13 | rs4986790 | TLR4- | homozygous | GCATACTTAGACTACTACCTCGATG[A] | SEQ ID |
| | | ANC | | TATTATTGACTTATTTAATTGTTTG | No: 13 |
| 14 | rs4986790 | TLR4- | heterozygous | GCATACTTAGACTACTACCTCGATG[A/G] | SEQ ID |
| | | HET | | TATTATTGACTTATTTAATTGTTTG | No: 14 |
| 15 | rs4986790 | TLR4- | homozygous | GCATACTTAGACTACTACCTCGATG[G] | SEQ ID |
| | | NONA | | TATTATTGACTTATTTAATTGTTTG | No: 15 |
| 16 | rs1205 | CRP- | homozygous | ACTTCCAGTTTGGCTTCTGTCCTCA[C] | SEQ ID |
| | | ANC | | AGTCTCTCTCCATGTGGCAAACAAG | No: 16 |
| 17 | rs1205 | CRP- | heterozygous | ACTTCCAGTTTGGCTTCTGTCCTCA[C/T] | SEQ ID |
| | | HET | | AGTCTCTCTCCATGTGGCAAACAAG | No: 17 |
| 18 | rs1205 | CRP- | homozygous | ACTTCCAGTTTGGCTTCTGTCCTCA[T] | SEQ ID |
| | | NONA | | AGTCTCTCTCCATGTGGCAAACAAG | No: 18 |
| 19 | rs4680 | COMT- | homozygous | CCAGCGGATGGTGGATTTCGCTGGC[G] | SEQ ID |
| | | ANC | | TGAAGGACAAGGTGTGCATGCCTGA | No: 19 |
| 20 | rs4680 | COMT- | heterozygous | CCAGCGGATGGTGGATTTCGCTGGC[A/G] | SEQ ID |
| | | HET | | TGAAGGACAAGGTGTGCATGCCTGA | No: 20 |
| 21 | rs4680 | COMT- | homozygous | CCAGCGGATGGTGGATTTCGCTGGC[A] | SEQ ID |
| | | NONA | | TGAAGGACAAGGTGTGCATGCCTGA | No: 21 |

*Unless otherwise indicated, the context sequences are in FASTA format, as presented by NCBI within the rs cluster report identified by “rs#” in the NCBI SNP reference database accessible at http://www.ncbi.nlm.nih.gov/snp.
**The naming conventions for the SNP Diploid Polymorphisms indicate the diploid is either - ANC (homozygous for the ancestral SNP), -HET (heterozygous as including one ancestral and one non-ancestral SNP in the diploid), or -NONA (homozygous for the non-ancestral SNP).
*** Brackets (i.e., “[. . .]”) appear within each context sequence to indicate the location (i.e., the “polymorphism marker” or “marker”) of the polymorphic region in the context sequence.
@Unless otherwise indicated, context sequences in FASTA format, are presented by NCBI within the rs cluster report identified by “rs#” associated with each rs number in Tables 1 above in the NCBI SNP reference database accessible at http://www.ncbi.nlm.nih.gov/snp, and which is incorporated by reference herein for each recited SNP rs number in the Table(s) above.

In Table 1, the active polymorphisms are the various diploid pair of alleles associated with “SNP markers” called “rs numbers” in the ref SNP database. Different diploid pairs for each allele have varying activities for generating prognostic information about NSAID mediated side effect risk. A SNP marker in dbSNP references a SNP cluster report identification number (i.e., the “rs number”) in the ref SNP database. The context sequences shown in Table 1 include the allelic variant(s) and the zygosity of the diploid pair identified as active for providing prognostic information according to the principles of the invention. The context sequences include the active polymorphism SNP located in the relevant region of the gene. The context sequences also include a number of nucleotide bases flanking the active polymorphism SNP in the relevant region of the gene. In the context sequences shown in Table 1, the polymorphic SNP location is shown in brackets within the context sequence for identification purposes. Table 1 also show the rs cluster report number (i.e., the “rs number”) associated with the active polymorphism SNP in dbSNP maintained by NCBI.

Studies have been conducted and it has been determined that SNP diploid polymorphisms identified in Table 1 are predictive of a differential predisposition to NSAID mediated side effect risk associated with a patient having one or more of SNP diploid polymorphisms. Select SNP diploid polymorphisms in Table 1 are associated with a patient having an elevated NSAID mediated side effect risk (i.e., predisposed to having a higher risk for NSAID-related side effects).

The test for NSAID mediated side effect risk has several categories. Each category is scored separately as shown in the charts below, but all are based on the following scoring system.

For diploid polymorphisms shown in Table 1 above, an exemplary scoring is shown Table 2 below:

TABLE 2
NSAID Risk Genetic Information Scoring

| | RS | ANC | ANC | HET | HET | NONA | NONA |
|---|---|---|---|---|---|---|---|
| GENE | Number | Def | Value | Def | Value | Def | Value |
| ABCB1 | rs1045642 | CC | 0 | CT | 0 | TT | 2 |
| COX1 | rs1330344 | GG | 2 | GA | 0 | AA | 0 |
| PTPN11 | rs2301756 | CC | 2 | CT | 2 | TT | 0 |
| NOD1 | rs7789045 | TT | 0 | TA | 0 | AA | 2 |
| TLR4 | rs4986790 | AA | 0 | AG | 2 | GG | 2 |
| CRP | rs1205 | CC | 0 | CT | 0 | TT | 2 |
| COMT | rs4680 | GG | 0 | GA | 0 | AA | 2 |

In addition, other CYPs having SNP diploid polymorphisms identified as also having a predisposition to NSAID mediated side effect risk are listed in Table 3 below. This profile includes an analysis of the enzymes CYP2C8 and CYP2C9, in which the presence of genetic coding variants indicates a risk factor for gastrointestinal hemorrhages associated with the use of NSAIDs due to a reduction in the enzymes' rate of metabolism. The risk profile combines the evaluation of relevant signalling cascades and metabolizing pathways to provide information regarding NSAID-induced risk factors for clinical use and management. Physicians may use this test to determine the likelihood of a patient experiencing an NSAID-related adverse event and/or to assist with prescribing NSAIDS at therapeutic doses.

Table 3—Identification and Grading of CYP SNP Haplotype Polymorphisms

TABLE 3A
CYP2C8 @
CYP2C8 Haplotype SNPs by Individual DNA Strand

| Haplotype Id | CYP2C8 | rs11572103 | rs10509681 | rs1058930 |
|---|---|---|---|---|
| PA165958681 | *1A | T | T | G |
| PA165958682 | *1B | T | T | G |
| PA165958683 | *1C | T | T | G |
| PA165958684 | *2 | A | T | G |
| PA165958685 | *3 | T | C | G |
| PA165958686 | *4 | T | T | C |
| PA165958687 | *5 | T | T | G |
| PA165958688 | *6 | T | T | G |
| PA165958689 | *7 | T | T | G |
| PA165958690 | *8 | T | T | G |
| PA165958691 | *9 | T | T | G |
| PA165958692 | *10 | T | T | G |
| PA165958693 | *11 | T | T | G |
| PA165958694 | *12 | T | T | G |
| PA165958695 | *13 | T | T | G |
| PA165958696 | *14 | T | T | G |

CYP2C8 Allele Pair Scoring

| SNP: | All. Pair ID: | Genotype | Star Alleles |
|---|---|---|---|
| rs10509681 | *3 | T/T | other/other |
| | | T/C | *3/other |
| | | C/C | *3/*3 |
| rs11572080 | *3 | C/C | *1/*1 |
| | | C/T | *3/*1 |
| | | T/T | *3/*3 |
| rs11572103 | *2 | T/T | other/other |
| | | T/A | *2/other |
| | | A/A | *2/*2 |
| rs1058930 | *4 | G/G | other/other |
| | | G/C | *4/other |
| | | C/C | *4/*4 |

CYP2C8: Allele Pair ID Scores

| NONE = 0 | DECREASED = 0.5 | NORMAL = 1 |
|---|---|---|
| *5 | *2 | *1A |
| | *3 | |
| | *4 | |

CYP2C8: Allele Pair ID Grade

| | |
|---|---|
| C | 1.0 = reduced/reduced |
| C | 1.5 = reduced/functional |
| B | 2 = functional/functional |

TABLE 3B
CYP2C9@

| CYP2C9 | rs1799853 | rs1057910 | rs28371686 | rs9332131 | rs7900194 | rs28371685 | rs72558187 |
|---|---|---|---|---|---|---|---|
| *1 | C | A | C | A | G | C | T |
| *2 | T | A | C | A | G | C | T |
| *3 | C | C | C | A | G | C | T |
| *4 | C | A | C | A | G | C | T |
| *5 | C | A | G | A | G | C | T |
| *6 | C | A | C | delA | G | C | T |
| *7 | C | A | C | A | G | C | T |
| *8 | C | A | C | A | A | C | T |
| *9 | C | A | C | A | G | C | T |
| *10 | C | A | C | A | G | C | T |
| *11 | C | A | C | A | G | T | T |
| *12 | C | A | C | A | G | C | T |
| *13 | C | A | C | A | G | C | C |
| *14 | C | A | C | A | G | C | T |
| *15 | C | A | C | A | G | C | T |
| *16 | C | A | C | A | G | C | T |
| *17 | C | A | C | A | G | C | T |
| *18 | C | C | C | A | G | C | T |
| *19 | C | A | C | A | G | C | T |
| *20 | C | A | C | A | G | C | T |
| *21 | C | A | C | A | G | C | T |
| *22 | C | A | C | A | G | C | T |
| *23 | C | A | C | A | G | C | T |
| *24 | C or T | A | C | A | G | C | T |
| *25 | C | A | C | A | G | C | T |
| *26 | C | A | C | A | G | C | T |
| *27 | C | A | C | A | G | C | T |
| *28 | C | A | C | A | G | C | T |
| *29 | C | A | C | A | G | C | T |
| *30 | C | A | C | A | G | C | T |
| *31 | C | A | C | A | G | C | T |
| *32 | C | A | C | A | G | C | T |
| *33 | C | A | C | A | G | C | T |
| *34 | C | A | C | A | G | C | T |
| *35 | T | A | C | A | G | C | T |
| *36 | C | A | C | A | G | C | T |
| *37 | C | A | C | A | G | C | T |
| *38 | C | A | C | A | G | C | T |
| *39 | C | A | C | A | G | C | T |
| *40 | C | A | C | A | G | C | T |
| *41 | C | A | C | A | G | C | T |
| *42 | C | A | C | A | G | C | T |
| *43 | C | A | C | A | G | C | T |
| *44 | C | A | C | A | G | C | T |
| *45 | C | A | C | A | G | C | T |
| *46 | C | A | C | A | G | C | T |
| *47 | C | A | C | A | G | C | T |
| *48 | C | A | C | A | G | C | T |
| *49 | C | A | C | A | G | C | T |
| *50 | C | A | C | A | G | C | T |
| *51 | C | A | C | A | G | C | T |
| *52 | C | A | C | A | G | C | T |
| *53 | C | A | C | A | G | C | T |
| *54 | C | A | C | A | G | C | T |
| *55 | C | A | C | A | G | C | T |
| *56 | C | A | C | A | G | C | T |
| *57 | C | A | C | A | G | C | T |
| *58 | C | A | C | A | G | C | T |

CYP2C9: Allele Scores

| NORMAL = 1 | NULL = 0 | INCREASED = 1.5 | DECREASED = 0.5 |
|---|---|---|---|
| *1A | *6 | | *3 |
| *1 | *35 in vitro | | *5 |
| | *15 | | *8 |
| | *25 | | *11 |
| | | | *13 |
| | | | *2 |
| | | | *18 |
| | | | *4 |
| | | | *12 |
| | | | *14 |
| | | | *16 |
| | | | *17 |
| | | | *33 |
| | | | *26 |
| | | | *28 |
| | | | *30 |
| | | | *33 |
| | | | *24 |

CYP2C9: Activity Scores

| | |
|---|---|
| D | 0 = null/null |
| D | 0.5 = null/reduced function |
| D | 1 = reduced/reduced OR normal/null |
| C | 1.5 = reduced/normal |
| B | 2 = normal/normal |
| A | >2 = more than 2 normal |

@ Unless otherwise indicated, context sequences in FASTA format, are presented by NCBI within the rs cluster report identified by “rs#” associated with each rs number in Tables 3A and 3B above in the NCBI SNP reference database accessible at http://www.ncbi.nlm.nih.gov/snp, and which is incorporated by reference herein for each recited SNP rs number in the Table(s) above.

For CYP haplotypes, with respect to NSAID risk assessment, an exemplary algorithm for determining NSAID mediated side effect risk is shown below. Each category is scored separately as shown in the charts below, but all are based on the following scoring system. As would be known by one of ordinary skill in the art, there are four general categories of CYP star alleles (i.e., CYP haplotypes): normal function, reduced function, null function and increased function. The nomenclature is reported by, for example, Robarge et al., “The Star-Allele Nomenclature: Retooling for Translational Genomics” Nature, v. 82, no. 3, September 2007, pp. 244-248, incorporated by reference herein.

A large number of star alleles have been reported for each cytochrome. Among these are normal functioning CYP star alleles, CYP star alleles with some function that is a reduced function, CYP star alleles with null (or non-functional) alleles, and CYP star alleles with increased functionality. These alleles convey a wide range of enzyme activity, from no activity to ultrarapid metabolism of substrates/medications. CYP2C8 is a B, according to Table 3, if two normal functional star alleles are detected (e.g. CYP2C8*1/*1) A normal functional star allele of CYP2C8 is CYP2C8*1 described above and in Table 3A.

CYP2C8 is a C, according to Table 3, if one normal functional and one reduced function star allele is detected (e.g. CYP2C8*1/*3, *1/*2, *1/*4, etc.), or if two reduced function star alleles are detected (e.g. CYP2C8*2/*2, *2/*3, *2/*4, *3/*3, *3/*4, *4/*4, etc.). Examples of reduced function star alleles of CYP2C8 are CYP2C8*2, *3, and *4.

CYP2C9 is a B according to Table 3 if two functional star alleles are detected (e.g. CYP2C9*1/*1). A normal functional star allele of CYP2C9 is CYP2C9*1 described above and in Table 3B. CYP2C9 is a C if one functional and one reduced function star allele is detected (e.g. CYP2C9*1/*2, *1/*3, *1/*5, *1/*8, *1/*11, *1/*13, *1/*18, *1/*24, etc.). Examples of reduced function star alleles of CYP2C9 are CYP2C9*2, *3, *5, *8, *11, *13, *18 and *24. CYP2C9 is a D if two reduced function star alleles are detected (e.g. any combination of *2,*3,*5,*8,*11,*13,*18,*24, etc.) or if two null function star alleles are detected (e.g. any combination of *6, *15, *25, *35, etc.), or if one null and one reduced function star allele is detected (e.g *2/*6, *3/*35, *6/*18, etc.), or if one functional and one nonfunctional star allele is detected (e.g. *1/*6, *1/*15, *1/*25, *1/*35, etc.). Examples of null function star alleles of CYP2C9 are CYP2C9*6, *15, *25 and *34.

The haplotypes and grading for the above mentioned CYP star alleles are also described in pending PCT Application No. TBD based on Attorney Docket No. P7916PC01 entitled “System and Method for Processing Genotype Information Relating to Drug Metabolism” by Brian Meshkin filed on Apr. 28, 2016, which is incorporated herein by reference in its entirety.

Cross grading and Drug Reccomendations shown in Table 4 and 6 below:

TABLE 4
Cross Grading

| | CYP2C9 | | | | |
|---|---|---|---|---|---|
| | | B | C | D | C/D |
| CYP2C8 | B | Not at risk | At risk | At risk | At risk |
| | B | Not at risk | At risk | At risk | At risk |

Using the Tables 2-4 above to score the raw results; then applying these scores to the Tables below to arrive at the interpretations reported on the tests.

NSAID Mediated Bleeding Ulcer Risk

NSAID MEDITATED BLEEDING ULCER RISK

| CATEGORY | Risk | COMMENTS |
|---|---|---|
| COX1 = 2 and | Predicted Risk | This patient is predicted to be at an elevated risk of |
| CYPs predict risk | | developing an NSAID-induced ulcer disease and |
| | | gastro-intestinal bleeding due to COX-1 and NSAID |
| | | metabolizing enzyme polymorphisms. Consider |
| | | treatment to reduce risk of gastric ulcers, such as |
| | | Protein Pump Inhibitors (PPIs) or Histamine H2- |
| | | receptor antagonists. Evaluating CYP2C19 and |
| | | CYP2D6 genetics, respectively, for these treatments |
| | | with the risk drug metabolism profile can help to |
| | | make the appropriate selection. |
| COX1 = 0 and | Some Predicted | This patient is predicted to have an elevated risk of |
| CYPs predict risk | Risk | developing acute gastro-intestinal bleeding due to |
| | | polymorphisms in NSAID metabolizing enzymes. |
| | | Consider treatment to reduce risk of gastric ulcers, |
| | | such as Protein Pump Inhibitors (PPIs) or Histamine |
| | | H2-receptor antagonists. Evaluating CYP2C19 and |
| | | CYP2D6 genetics, respectively, for these treatments |
| | | with the risk drug metabolism profile can help to |
| | | make the appropriate selection. |
| COX1 = 2 and | Some Predicted | This patient is predicted to be at an elevated risk of |
| CYPs do not | RIsk | developing an NSAID-induced ulcer disease due to |
| predict risk | | COX-1 polymorphisms. Inhibition of the Cox-1 |
| | | enzyme by non-selective NSAIDs reduces the |
| | | synthesis of prostaglandins, which promote |
| | | inflammation, but also play a protective role in the |
| | | gastrointestinal tract. Consideration of a Cox-2 |
| | | selective NSAID treatment may result in better |
| | | clinical outcomes. |
| COX1 = 0 and | No Predicted | This patient is not predicted to be at an increased |
| CYPs do not | Risk | risk of developing an NSAID-induced ulcer or |
| predict risk | | gastro-intestinal complications. |

NSAID Mediated Cardiovascular Risk

To assess cardiovascular risk, the NSAID risk test evaluates the Cox-1 gene and the CRP gene, a marker of inflammation and a predictor of cardiovascular risk. Cardiovascular risk appears to be due, at least in part, to disequilibrium in prostaglandin synthesis between pro-thrombotic thromboxane A2 and anti-thrombotic prostacyclin, both of which are regulated by COX enzymes. However, because the use of low-dose aspirin does not appear to attenuate the risk of cardiovascular events, it is suggested that only some NSAID users are genetically susceptible to increased risk. Thus, a gene-drug interaction appears to modulate this cardiovascular risk through prostaglandin synthesis or other inflammatory pathways.

Additionally, a Val/Met polymorphism (r54860) in the catechol-O-methyltransferase (COMT) gene, which codes for an enzyme that catabolizes catecholamines such as dopamine, epinephrine, and norepinephrine, is implicated in NSAID-induced cardiovascular risk. COMT is present in platelets and in endothelial and vascular smooth muscle cells, where the attenuated COMT activity of the MET allele homozygotes could increase catecholamine flux and oxidant stress, thus lowering the threshold for platelet activation and endothelial dysfunction.

NSAID MEDITATED CARDIOVASCULAR RISK

| CATEGORY | Risk | COMMENTS |
|---|---|---|
| If CRP = 2 and | Predicted Risk | This patient is predicted to be at an increased risk of |
| COX1 = 2 | | developing an acute coronary syndrome with |
| | | concomitant NSAID use. Gene variants in key |
| | | inflammatory and prostaglandin metabolism |
| | | pathways have been shown to lead to different |
| | | levels of cardiovascular risk with NSAID treatment. |
| | | Consider appropriate pharmaceutical agents to |
| | | lower C-Reactive Protein levels, such as statins or |
| | | thiazolidinediones |
| If CRP = 0 and | Some Predicted Risk | This patient has a COX-1 genetic variant that is |
| COX1 = 2 | | predicted to be ineffectively regulated with aspirin |
| | | treatment. If taking aspirin, this patient is at an |
| | | increased risk of experiencing an adverse |
| | | cardiovascular event due to aspirin resistance. |
| | | Consider alternatives to aspirin for heart disease and |
| | | stroke prevention. |
| If CRP = 2 and | Some Predicted | This patient is predicted to be at an increased risk of |
| COX1 = 0 | Risk | developing an acute coronary syndrome with |
| | | concomitant NSAID use. Gene variants in key |
| | | inflammatory and prostaglandin metabolism |
| | | pathways have been shown to lead to different |
| | | levels of cardiovascular risk with NSAID treatment. |
| | | Consider appropriate pharmaceutical agents to |
| | | lower C-Reactive Protein levels, such as statins, |
| | | aspirin, or thiazolidinediones |
| If CRP = 0 and | No Predicted | This patient is not predicted to be at an increased |
| COX1 = 0 | Risk | risk of developing an acute coronary syndrome with |
| | | concomitant NSAID use. |
| If COMT | No Predicted | In general, this patient carries a COMT genotype |
| rs4680 = G/G | Risk | that confers a lower risk of Coronary Artery Disease |
| (valine) | | and cardiovascular disease. Preventative treatment |
| | | with aspirin has not been shown to reduce incident |
| | | cardiovascular disease in patients with this |
| | | genotype. |
| If COMT | Some Predicted | In general, this patient carries a COMT genotype |
| rs4680 = A/A | Risk | that confers a higher risk of Coronary Artery |
| (Methionine) | | Disease and cardiovascular disease. However, |
| | | treatment with aspirin and Vitamin E has been |
| | | shown to prevent incident cardiovascular disease in |
| | | patients with this genotype. |
| If COMT | Some Predicted | In general, this patient carries a COMT genotype |
| rs4680 = G/A | Risk | that confers a moderate risk of Coronary Artery |
| (Val/Met) | | Disease and cardiovascular disease. However, |
| | | preventative treatment with aspirin has not been |
| | | shown to reduce incident cardiovascular disease in |
| | | patients with this genotype. |

NSAID Mediated Aspirin Resistance Risk
COX-1 SNP rs1330344 is implicated in “Aspirin resistance.” The antiplatelet effects of aspirin may not be equal in all individuals; therefore, a proportion of patients prescribed aspirin suffer recurrent thromboembolic vascular events, giving rise to the term “aspirin resistance.” This risk is increased in COX-1 rs1330344 GG homozygotes and in ABCB1 TT homozygotes as indicated above in Table 2.

NSAID MEDITATED ASPIRIN RESISTANCE RISK

| CATEGORY | Risk | COMMENTS |
|---|---|---|
| If ABCB1 = 2 and | Predicted Risk | This patient is predicted to be at an increased risk of |
| COX1 = 2 | | aspirin resistance. Consider an alternative to aspirin |
| | | for prevention of heart disease and stroke. Aspirin |
| | | inhibits platelet activation and aggregation via |
| | | multifactoral mechanisms - including those |
| | | determined by heritable factors, such as variants in |
| | | multi-drug resistance efflux pumps and COX1 |
| | | genes. Aspirin resistance refers to an absence of an |
| | | expected pharmacological effect and/or poor |
| | | clinical outcomes, such as recurrent vascular events. |
| If ABCB1 = 0 | Some Predicted | This patient is predicted to be at an increased risk of |
| and COX1 = 2 | R [symbol] isk | aspirin resistance. Consider an alternative to aspirin |
| | | for prevention of heart disease and stroke. Aspirin |
| | | inhibits platelet activation and aggregation via |
| | | multifactoral mechanisms - including those |
| | | determined by heritable factors, such as variants in |
| | | multi-drug resistance efflux pumps and COX1 |
| | | genes. Aspirin resistance refers to an absence of an |
| | | expected pharmacological effect and/or poor |
| | | clinical outcomes, such as recurrent vascular events. |
| If ABCB1 = 2 | Some Predicted | This patient is predicted to be at an increased risk of |
| and COX1 = 0 | Risk | aspirin resistance. Consider an alternative to aspirin |
| | | for prevention of heart disease and stroke. Aspirin |
| | | inhibits platelet activation and aggregation via |
| | | multi-factoral mechanisms - including those |
| | | determined by heritable factors, such as variants in |
| | | multi-drug resistance efflux pumps and COX1 |
| | | genes. Aspirin resistance refers to an absence of an |
| | | expected pharmacological effect and/or poor |
| | | clinical outcomes, such as recurrent vascular events. |
| If ABCB1 = 0 | No Predicted | This patient is not predicted to be at an increased |
| and COX1 = 0 | Risk | risk of aspirin resistance. Aspirin inhibits platelet |
| | | activation and aggregation via multifactoral |
| | | mechanisms - including those determined by |
| | | heritable factors, such as variants in multi-drug |
| | | resistance efflux pumps and COX1 genes. Aspirin |
| | | resistance refers to an absence of an expected |
| | | pharmacological effect and/or poor clinical |
| | | outcomes, such as recurrent vascular events. |

NSAID Mediated H. Pylori Gastropathies Risk [symbol]
indicates data missing or illegible when filed

NSAID testing provides a report and interpretation for the association of polymorphisms with gastro-intestinal risks with concomitant NSAID use. For example, Cox-1 rs1330344 is associated with the development of NSAID-induced ulcer diseases. In addition, a SNP in the TLR4 gene is associated with increased risk of development of premalignant gastric abnormalities of hypochlorhydria and atrophy, and also the risk of noncardia gastric carcinoma.

NSAID MEDITATED H. PYLORI RISK

| CATEGORY | Risk | COMMENTS |
|---|---|---|
| NOD1 = 2 and | Predicted Risk | This patient is predicted to be at an elevated risk |
| PTP = 2 and | | of developing gastric atrophy and/or cancer after |
| TLR = 2 | | Helibacter pylori infection. Consider alternatives |
| | | to NSAID therapies to mitigate the risk of |
| | | gastropathies. the risk of gastropathies. |
| NOD1 = 2 and | Some Predicted | This patient is predicted to be at an elevated risk |
| (PTP + TLR) = 2 | Risk | of developing gastric atrophy and/or cancer after |
| | | Helibacter pylori infection. Consider alternatives |
| | | to NSAID therapies to mitigate the risk of |
| | | gastropathies. |
| NOD1 = 2 and | Some Predicted | This patient is predicted to be at an elevated risk |
| PTP = 0 and | Risk | of developing gastric atrophy and/or cancer after |
| TLR = 0 | | Helibacter pylori infection. Consider alternatives |
| | | to NSAID therapies to mitigate the risk of |
| | | gastropathies. |
| NOD1 = 0 and | Some Predicted | This patient is predicted to be at an elevated risk |
| PTP = 2 and | Risk | of developing gastric atrophy and/or cancer after |
| TLR = 2 | | Helibacter pylori infection. Consider alternatives |
| | | to NSAID therapies to mitigate the risk of |
| | | gastropathies. |
| NOD1 = 0 and | Some Predicted | This patient is predicted to be at an elevated risk |
| PTP = 2 and | Risk | of developing gastric atrophy after Helibacter |
| TLR = 0 | | pylori infection. Consider alternatives to NSAID |
| | | therapies to mitigate the risk of gastropathies. |
| NOD1 = 0 and | Some Predicted | This patient is predicted to be at an elevated risk |
| TLR = 2 | Risk | of developing gastric atrophy and/or cancer after |
| | | Helibacter pylori infection. Consider |
| | | alternatives to NSAID therapies to mitigate the |
| | | risk of gastropathies. |
| NOD1 = 0 and | No Predicted Risk | This patient is not predicted to be at an |
| (PTP + TLR) = 0 | | increased risk of developing gastropathies with |
| | | concomitant NSAID use. |

Cytochrome Enzymes CYP2C8 and CYP2C9

The existence of genetic polymorphisms in metabolizing enzymes can be regarded as one of the principal causes of inter-individual variation in response to medications and in development of adverse reactions. In the case of cytochrome enzymes CYP2C8 and CYP2C9, the presence of genetic coding variants could be considered a risk factor for suffering from gastrointestinal hemorrhages associated with the use of NSAIDs, due to a reduction in the enzymes' rate of metabolism. The NSAID risk profile includes a combinatorial evaluation of CYP2C8 and CYP2C9 to provide a more comprehensive understanding of NSAIDs metabolism and associated risks, as shown in the above tables.

NSAID risk assessment relies on non-invasive measures of biological pathways that correlate with gastro-intestinal risk. The use of pharmacogenetic testing provides a quick and easy evaluation of genetic risk associated with NSAID use, in addition to providing an avenue for identification of new measures that may lead to increased accuracy in patient risk stratification. With a simple buccal swab, the risk test investigates potential gene-drug interactions analyzing prostaglandin synthesis enzyme targets of NSAIDs, inflammation, and other cardiovascular disease processes. A human sample that provides genomic DNA is acceptable for this test; examples are: buccal swabs, blood, urine, or tissue samples.

Using this approach, guidance for the rational use of NSAID therapy and clinical protocals can be achieved. For example, by identifying patients more likely to be good vs. poor responders; and providing alternative measures to control pain in patients with a poor likelihood of response. Alternative pain control measures to be considered based on the results of this test may lead to better patient outcomes, decreased use of suboptimal medications, and shorter duration of therapy and lower costs. Additionally, a characterization of a patient's metabolic profile for NSAID-induced hepatotoxicity would add crucial information to a patient's clinical care as well.

Detection of point mutations or other types of the allelic variants in Tables 1 and 3 can be accomplished several ways known in the art, such as by molecular cloning of the specified allele and subsequent sequencing of that allele using techniques known in the art. Alternatively, the gene sequences can be amplified directly from a genomic DNA preparation from the DNA sample using PCR, and the sequence composition is determined from the amplified product. As described more fully below, numerous methods are available for analyzing a subject's DNA for mutations at a given genetic locus such as the gene of interest.

One such detection method is allele specific hybridization using probes overlapping the polymorphic region and having, for example, about 5, or alternatively 10, or alternatively 20, or alternatively 25, or alternatively 30 nucleotides around the polymorphic region. In another embodiment, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described, e.g., in Cronin et al. (1996) Human Mutation 7:244.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

If the polymorphic region is located in the coding region of the gene of interest, yet other methods than those described above can be used for determining the identity of the allelic variant according to methods known in the art.

The genotype information obtained from analyzing a sample of a patient's genetic material may be utilized, according to the principles of the invention, to predict whether a patient has a level of risk associated with NSAID mediated side effect. The risk may be associated with a side effect the patient may be susceptible to developing, an efficacy of the drug to the patient specifically or some combination thereof. The genotype information of the patient may be combined with demographic information about the patient as described above.

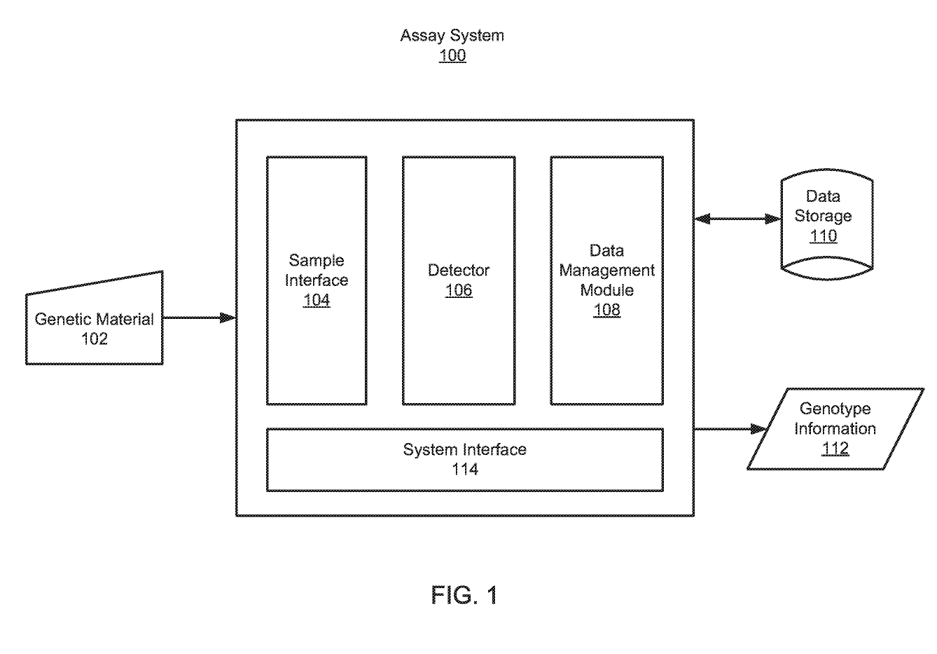

Referring to FIG. 1, depicted is an assay system 100. An assay system, such as assay system 100, may access or receive a genetic material, such as genetic material 102. The sample of genetic material 102 can be obtained from a patient by any suitable manner. The sample may be isolated from a source of a patient's DNA, such as saliva, buccal cells, hair roots, blood, cord blood, amniotic fluid, interstitial fluid, peritoneal fluid, chorionic villus, semen, or other suitable cell or tissue sample. Methods for isolating genomic DNA from various sources are well-known in the art. Also contemplated are non-invasive methods for obtaining and analyzing a sample of genetic material while still in situ within the patient's body.

The genetic material 102 may be received through a sample interface, such as sample interface 104 and detected using a detector, such as detector 106. A polymorphism may be detected in the sample by any suitable manner known in the art. For example, the polymorphism can be detected by techniques, such as allele specific hybridization, allele specific oligonucleotide ligation, primer extension, minisequencing, mass spectroscopy, heteroduplex analysis, single strand conformational polymorphism (SSCP), denaturing gradient gel electrophoresis (DGGE), oligonucleotide microarray analysis, temperature gradient gel electrophoresis (TGGE), and combinations thereof to produce an assay result. The assay result may be processed through a data management module, such as data management module 108, to produce genotype information 112. The genotype information 112 may include an assay result on whether the patients has a genotype including one or more of the allelic variants listed in Tables I and 3 above. The genotype information 112 may be stored in data storage 110 or transmitted to another system or entity via a system interface 114.

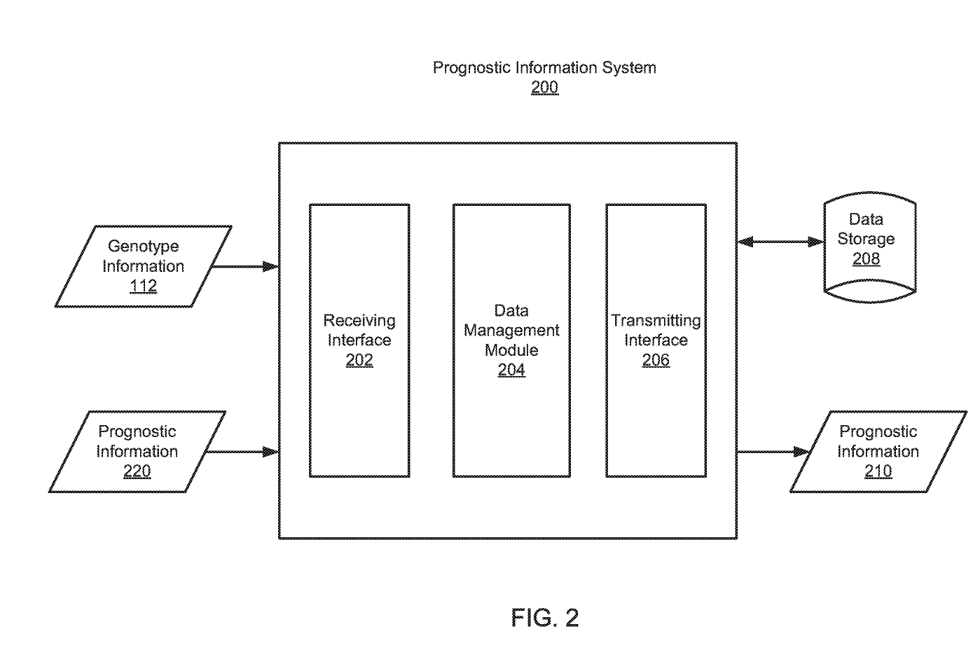

Referring to FIG. 2, depicted is a prognostic information system 200. The prognostic information system 200 may be remotely located away from the assay system 100 or operatively connected with it in an integrated system. The prognostic information system 200 receives the genotype information 112 through a receiving interface 202 for processing at a data management module 204 to generate prognostic information 210. The data management module 204 may utilize one or more algorithms described in greater detail below to generate prognostic information 210. The prognostic information 210 may be stored in data storage 208 or transmitted via a transmitting interface 206 to another system or entity. The transmitting interface 206 may be the same or different as the receiving interface 202. Furthermore, the system 200 may receive prognostic information 220 prepared by another system or entity. Prognostic information may be utilized, in addition to or in the alternative, to genotype information 112 in generating prognostic information 210.

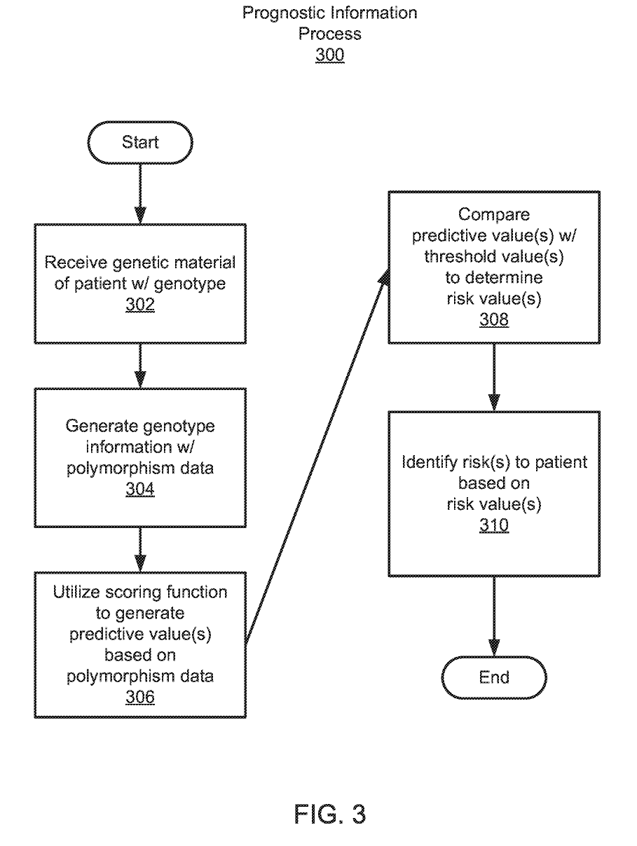

Referring to FIG. 3, depicted is a prognostic information process 300 which may be utilized for preparing information, such as genotype information 112 and prognostic information 210, utilizing an assay system, such as assay system 100 and/or a prognostic information system, such as prognostic information system 200, according to an embodiment. The steps of process 300, and other methods described herein, are described by way of example with the assay system 100 and the prognostic information system 200. The process 300 may be performed with other systems as well.

After process start, at step 302, a sample of genetic material of a patient is obtained as it is received at the sample interface 106. The sample interface can be any type of receptacle for holding or isolating the genetic material 102 for assay testing.

At step 304, the genetic material 102 is tested utilizing the detector 106 in assay system 100 to generate genotype information 112. The detector 106 may employ any of the assay methodologies described above to identify allelic variants in the genetic material 102 and generate the genotype information 112 including polymorphism data associated with one or more of the DNA polymorphisms described above in Tables 1 and 3. The data management module 108, utilizing a processor in an associated platform such as described below, may store the genotype information 112 on the data storage 110 and/or transmit the genotype information 112 to another entity or system, such as prognostic information system 200 where it is received at receiving interface 202 for analysis.

At step 306, the genotype information 112 can be analyzed utilizing a processor in an associated platform, such as described below, by using an algorithm which may be programmed for processing through data management module 204. The algorithm may utilize a scoring function to generate predictive values based on the polymorphism data in the genotype information 112. Different algorithms may be utilized to assign predictive values and aggregate values.

For example, an additive effect algorithm may be utilized to generate an analysis of a patient's genetic predisposition and their demographic phenotype predisposition to NSAID mediated side effect risk. In the additive effect algorithm, polymorphism data of the genotype information obtained from analyzing a patient's genetic material is utilized to indicate the active polymorphisms identified from a patient's genotype information. A tested polymorphism may be determined to be (1) absent or present in either (2) a heterozygous or (3) a homozygous variant in the patient's genotype. According to the additive effect algorithm, the polymorphisms identified from a patient's genotype information and demographic phenotype are each assigned a real value, such as an Odds Ratio (OR) or a parameter score, depending on which polymorphisms appears in the patient's genotype and demographic information.

To gather data for the algorithm, one or more of the SNP Diploid Polymorphisms, such as those listed in Tables 1 and 3, may be tested and/or analyzed to produce one or more values associated with the presence or absence of the SNP Diploid Polymorphisms. Other factors, such as other SNP Diploid Polymorphisms, other demographic phenotypes may also be tested and/or analyzed to produce one or more values associated with the presence or absence of the other SNP Diploid Polymorphisms and other demographic phenotypes.

The values gathered are based on results of the various tests and data gathered and/or determined. The values may be factored into an algorithm to score a subject's risk of NSAID mediated side effect based on the subject's genetic information and/or non-genetic characteristics or phenotypes. The algorithm may compute a composite score based on the results of individual tests. The composite score may be calculated based on an additive analysis of the individual scores which may be compared with a threshold value for determining NSAID mediated side effect risk based on the additive score. In addition or in the alternative, more complex functions may be utilized to process the values developed from the testing results, such as utilizing one or more weighting factor(s) applied to one or more of the individual values based on various circumstances, such as if a subject was tested using specific equipment, a temporal condition, etc.

In all of the preceding examples, the predictive values and aggregate values generated are forms of prognostic information 210.

At step 310, the result of the comparison obtained in step 308 generates a second form of prognostic information 220. For example, (a) if the determined sum is higher than the threshold value, it can be predicted that the patient is at an elevated risk for NSAID mediated side effect risk associated with prescribing the patient a NSAID medication; (b) if the determined sum is at or near the threshold value, it can be predicted that the patient is at a moderate risk for NSAID mediated side effect; and (c) if the determined sum is below the threshold value, it can be predicted that the patient is at a low risk for NSAID mediated side effect.

Also at step 310, the data management module 205 in the prognostic information system 200 identifies a risk to a patient by executing an algorithm, such as the additive effect algorithm described above, and communicating the generated prognostic information 210. The data management module 204, utilizing a processor in an associated platform such as described below, may store the prognostic information 210 on the data storage 208 and/or transmit the prognostic information 210 to another entity or system prior to end of the prognostic information process 300. Other algorithms may also be used in a similar manner to generate useful forms of prognostic information for determining treatment options for a patient.

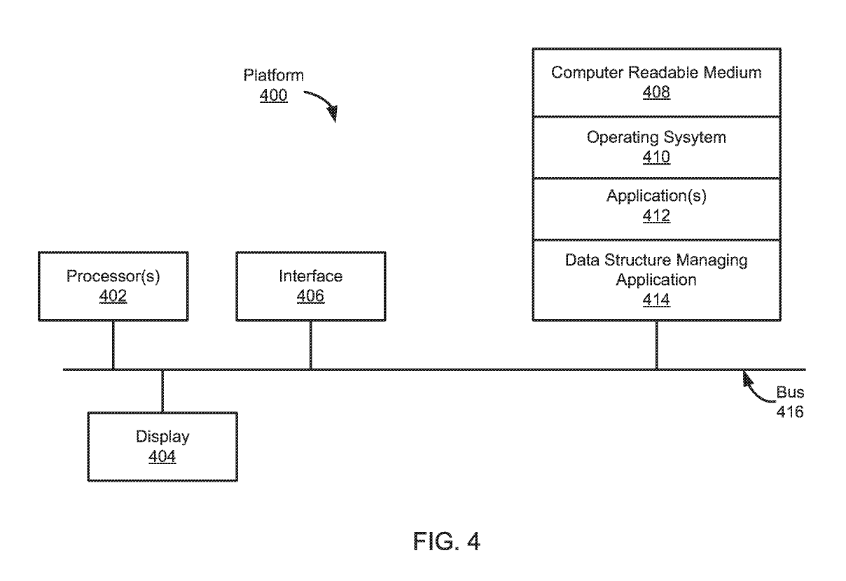

Referring to FIG. 4, there is shown a platform 400, which may be utilized as a computing device in a prognostic information system, such as prognostic information system 200, or an assay system, such as assay system 100. It is understood that the depiction of the platform 400 is a generalized illustration and that the platform 400 may include additional components and that some of the components described may be removed and/or modified without departing from a scope of the platform 400.

The platform 400 includes processor(s) 402, such as a central processing unit; a display 404, such as a monitor; an interface 406, such as a simple input interface and/or a network interface to a Local Area Network (LAN), a wireless 802.11x LAN, a 3G or 4G mobile WAN or a WiMax WAN; and a computer-readable medium (CRM) 408. Each of these components may be operatively coupled to a bus 416. For example, the bus 416 may be an EISA, a PCI, a USB, a FireWire, a NuBus, or a PDS.

A CRM, such as CRM 408 may be any suitable medium which participates in providing instructions to the processor(s) 402 for execution. For example, the CRM 408 may be non-volatile media, such as an optical or a magnetic disk; volatile media, such as memory; and transmission media, such as coaxial cables, copper wire, and fiber optics. Transmission media can also take the form of acoustic, light, or radio frequency waves. The CRM 408 may also store other instructions or instruction sets, including word processors, browsers, email, instant messaging, media players, and telephony code.

The CRM 408 may also store an operating system 410, such as MAC OS, MS WINDOWS, UNIX, or LINUX; application(s) 412, such as network applications, word processors, spreadsheet applications, browsers, email, instant messaging, media players such as games or mobile applications (e.g., “apps”); and a data structure managing application 414. The operating system 410 may be multi-user, multiprocessing, multitasking, multithreading, real-time and the like. The operating system 410 may also perform basic tasks such as recognizing input from the interface 406, including from input devices, such as a keyboard or a keypad; sending output to the display 404 and keeping track of files and directories on CRM 408; controlling peripheral devices, such as disk drives, printers, image capture devices; and for managing traffic on the bus 416. The applications 412 may include various components for establishing and maintaining network connections, such as code or instructions for implementing communication protocols including those such as TCP/IP, HTTP, Ethernet, USB, and FireWire.

A data structure managing application, such as data structure managing application 414 provides various code components for building/updating a computer-readable system architecture, such as for a non-volatile memory, as described above. In certain examples, some or all of the processes performed by the data structure managing application 412 may be integrated into the operating system 410. In certain examples, the processes may be at least partially implemented in digital electronic circuitry, in computer hardware, firmware, code, instruction sets, or any combination thereof.

Although described specifically throughout the entirety of the disclosure, the representative examples have utility over a wide range of applications, and the above discussion is not intended and should not be construed to be limiting. The terms, descriptions and figures used herein are set forth by way of illustration only and are not meant as limitations. Those skilled in the art recognize that many variations are possible within the spirit and scope of the principles of the invention. While the examples have been described with reference to the figures, those skilled in the art are able to make various modifications to the described examples without departing from the scope of the following claims, and their equivalents.

1. A method comprising facilitating a processing of and/or processing (1) data and/or (2) information and/or (3) at least one signal, the (1) data and/or (2) information and/or (3) at least one signal based, at least in part, on the following:
determining patient information, including DNA information, associated with a human subject;
determining from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group:
ABCB1-ANC, ABCB1-HET, and ABCB1-NONA in the ABCB1 gene, COX1-ANC, COX1-HET, and COX1-NONA in the COX1 gene, PTPN11-ANC, PTPN11-HET, and PTPN11-NONA in the PTPN11 gene, NOD1-ANC, NOD1-HET, and NOD1-NONA in the NOD1 gene, TLR4-ANC, TLR4-HET, and TLR4-NONA in the TLR4 gene, CRP-ANC, CRP-HET, and CRP-NONA in the CRP gene, and COMT-ANC, COMT-HET, and COMT-NONA in the COMT gene; and
determining a nonsteroidal anti-inflammatory drug (NSAID) mediated side effect risk associated with the human subject based, at least in part, on the presence or absence of the one or more SNP diploid polymorphisms in the subject genotype.

2. A method of claim 1, wherein the (1) data and/or (2) information and/or (3) at least one signal are further based, at least in part, on the following:
determining from the DNA information whether a subject genotype of the human subject includes at least two CYP haplotype polymorphisms by detecting, utilizing a detection technology and the DNA information, a presence or absence of the at least two CYP haplotype polymorphisms in the subject genotype,
wherein at least one or more CYP haplotype polymorphisms are selected from the CYP2C8 haplotype group including normal function CYP2C8 star alleles and reduced function CYP2C8 star alleles,
wherein at least one or more CYP haplotype polymorphisms are selected from the CYP2C9 haplotype group including normal function CYP2C9 star alleles, reduced function CYP2C9 star alleles and null function CYP29 star alleles.

3. A method of claim 1, wherein the (1) data and/or (2) information and/or (3) at least one signal are further based, at least in part, on the following:
determining a comparing of a region, including the one or more SNP diploid polymorphisms, of the subject genotype with a corresponding region of a predetermined reference genotype, wherein characteristics of the corresponding region of the reference genotype are based upon a predetermined population norm;
determining prognostic information associated with the human subject based on the determined NSAID mediated side effect risk; and
determining a therapy for the human subject based on the determined prognostic information associated with the human subject,
wherein the method for determining the NSAID risk associated with the human subject, is an ex vivo method.

4. A method of claim 1, wherein the one or more SNP diploid polymorphisms include at least three SNP diploid polymorphisms from the SNP diploid group.

5. A method of claim 1, wherein the one or more SNP diploid polymorphisms include at least four SNP diploid polymorphisms from the SNP diploid group.

6. A method of claim 1, wherein the one or more SNP diploid polymorphisms include at least five SNP diploid polymorphisms from the SNP diploid group.

7. A method of claim 1, wherein the one or more SNP diploid polymorphisms include at least seven SNP diploid polymorphisms from the SNP diploid group.

8. An apparatus comprising:
at least one processor; and
at least one memory including computer program code for one or more programs,
the at least one memory and the computer program code configured to, with the at least one processor, cause the apparatus to perform at least the following,
determine patient information, including DNA information, associated with a human subject;
determine from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group:
ABCB1-ANC, ABCB1-HET, and ABCB1-NONA in the ABCB1 gene, COX1-ANC, COX1-HET, and COX1-NONA in the COX1 gene, PTPN11-ANC, PTPN11-HET, and PTPN11-NONA in the PTPN11 gene, NOD1-ANC, NOD1-HET, and NOD1-NONA in the NOD1 gene, TLR4-ANC, TLR4-HET, and TLR4-NONA in the TLR4 gene, CRP-ANC, CRP-HET, and CRP-NONA in the CRP gene, and COMT-ANC, COMT-HET, and COMT-NONA in the COMT gene; and
determine a nonsteroidal anti-inflammatory drug (NSAID) mediated side effect risk associated with the human subject based, at least in part, on the presence or absence of the one or more SNP diploid polymorphisms in the subject genotype.

9. An apparatus of claim 8, wherein the (1) data and/or (2) information and/or (3) at least one signal are further based, at least in part, on the following:
determining from the DNA information whether a subject genotype of the human subject includes at least two CYP haplotype polymorphisms by detecting, utilizing a detection technology and the DNA information, a presence or absence of the at least two CYP haplotype polymorphisms in the subject genotype,
wherein at least one or more CYP haplotype polymorphisms are selected from the CYP2C8 haplotype group including normal function CYP2C8 star alleles and reduced function CYP2C8 star alleles,
wherein at least one or more CYP haplotype polymorphisms are selected from the CYP2C9 haplotype group including normal function CYP2C9 star alleles, reduced function CYP2C9 star alleles and null function CYP29 star alleles.

10. An apparatus of claim 8, wherein the apparatus is further caused to:
determine a comparing of a region, including the one or more SNP diploid polymorphisms, of the subject genotype with a corresponding region of a predetermined reference genotype,
wherein characteristics of the corresponding region of the reference genotype are based upon a predetermined population norm;
determine prognostic information associated with the human subject based on the determined NSAID mediated side effect risk; and
determine a therapy for the human subject based on the determined prognostic information associated with the human subject,
wherein the methodology for determining the NSAID risk associated with the human subject associated with the apparatus, is an ex vivo methodology.

11. An apparatus of claim 8, wherein the one or more SNP diploid polymorphisms include at least three SNP diploid polymorphisms from the SNP diploid group.

12. An apparatus of claim 11, wherein the one or more SNP diploid polymorphisms include at least four SNP diploid polymorphisms from the SNP diploid group.

13. An apparatus of claim 8, wherein the one or more SNP diploid polymorphisms include at least five SNP diploid polymorphisms from the SNP diploid group.

14. An apparatus of claim 8, wherein the one or more SNP diploid polymorphisms include at least seven SNP diploid polymorphisms from the SNP diploid group.

15. A non-transitory computer readable medium storing computer readable instructions that when executed by at least one processor perform a method, the method comprising facilitating a processing of and/or processing (1) data and/or (2) information and/or (3) at least one signal, the (1) data and/or (2) information and/or (3) at least one signal based, at least in part, on the following:
determining patient information, including DNA information, associated with a human subject;
determining from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group:
ABCB1-ANC, ABCB1-HET, and ABCB1-NONA in the ABCB1 gene, COX1-ANC, COX1-HET, and COX1-NONA in the COX1 gene, PTPN11-ANC, PTPN11-HET, and PTPN11-NONA in the PTPN11 gene, NOD1-ANC, NOD1-HET, and NOD1-NONA in the NOD1 gene, TLR4-ANC, TLR4-HET, and TLR4-NONA in the TLR4 gene, CRP-ANC, CRP-HET, and CRP-NONA in the CRP gene, and COMT-ANC, COMT-HET, and COMT-NONA in the COMT gene; and
determining a nonsteroidal anti-inflammatory drug (NSAID) mediated side effect risk associated with the human subject based, at least in part, on the presence or absence of the one or more SNP diploid polymorphisms in the subject genotype.

16. A computer readable medium of claim 15, wherein the (1) data and/or (2) information and/or (3) at least one signal are further based, at least in part, on the following:
determining from the DNA information whether a subject genotype of the human subject includes at least two CYP haplotype polymorphisms by detecting, utilizing a detection technology and the DNA information, a presence or absence of the at least two CYP haplotype polymorphisms in the subject genotype,
wherein at least one or more CYP haplotype polymorphisms are selected from the CYP2C8 haplotype group including normal function CYP2C8 star alleles and reduced function CYP2C8 star alleles,
wherein at least one or more CYP haplotype polymorphisms are selected from the CYP2C9 haplotype group including normal function CYP2C9 star alleles, reduced function CYP2C9 star alleles and null function CYP29 star alleles.

17. A computer readable medium of claim 15, wherein the (1) data and/or (2) information and/or (3) at least one signal are further based, at least in part, on the following:
determining a comparing of a region, including the one or more SNP diploid polymorphisms, of the subject genotype with a corresponding region of a predetermined reference genotype,
wherein characteristics of the corresponding region of the reference genotype are based upon a predetermined population norm;
determining prognostic information associated with the human subject based on the determined NSAID mediated side effect risk; and
determining a therapy for the human subject based on the determined prognostic information associated with the human subject.
wherein the methodology for determining the opioid dependency risk associated with the human subject associated with the computer readable medium, is an ex vivo methodology.

18. A computer readable medium of claim 15, wherein the one or more SNP diploid polymorphisms include at least three SNP diploid polymorphisms from the SNP diploid group.

19. A computer readable medium of claim 15, wherein the one or more SNP diploid polymorphisms include at least five SNP diploid polymorphisms from the SNP diploid group.

20. A computer readable medium of claim 15, wherein the one or more SNP diploid polymorphisms include at least seven SNP diploid polymorphisms from the SNP diploid group.
